(19) United States
(12) Patent Application Publication
(10) Pub. No.: US 2022/0202692 A1
(43) Pub. Date: Jun. 30, 2022

(54) NATURAL HYDRATING MOISTURIZER HAIR THERAPY OIL

(71) Applicant: Lania Kashun Collins, Los Angeles, CA (US)

(72) Inventor: Lania Kashun Collins, Los Angeles, CA (US)

(21) Appl. No.: 17/247,941

(22) Filed: Dec. 31, 2020

(51) Int. Cl. A61K 8/92; A61K 8/9789; A61Q 5/12

(52) U.S. Cl. CPC A61K 8/922; A61K 8/9789; A61K 2800/805; A61K 2800/591; A61Q 5/12

(57) ABSTRACT

A natural hydrating moisturizer hair therapy oil having as components coconut oil, tea tree, organic black castor oil, biotin, argon oil, and Jamaican mango and lime black castor oil extra dark.

### BACKGROUND OF THE INVENTION

The present invention relates to hair treatment systems and methods and, more particularly, a hair therapeutic system embodied in a natural hydrating moisturizer hair therapy oil.

Proper hair treatment is important to maintain healthy hair and prevent damage that might lead to further problems. Unfortunately, unhealthy hair is universal to all cultures of hair, even babies' hair suffers from dry scalp (“cradle cap”). Adult hair is also susceptible to dry scalp and resulting hair loss, hair frizz, hair dullness and hair hardness, which increases the probability of hair breakage.

Current hair treatment systems do not work due to their reliance on alcohol, which causes breakage, dries hair and dries hair follicles.

As can be seen, there is a need for a hair therapeutic system embodied in a natural hydrating moisturizer hair therapy oil that solves dry scalp, breakage and hair loss, as well as tames hair frizz and increases the shine and softness of hair. The present invention enables moisture and hydration to the hair and the hair follicles through improving blood circulation flow to the scalp, which in turn nourishes hair roots, facilitating fast regrowth of thicker hair. Moreover, the result of the hair therapy of the present invention increases hair elasticity and restores lifeless hair. The hair therapy oil embodied in the present invention is an improvement over the prior art because it is multiple hair treatments in one bottle.

Furthermore, the hair therapy oil is light and easy to use and produces quick improvements users can see and feel. The hair therapy oil helps regrow hair wherever a user desires through an all-natural product that protects the hair from being damaged and that allows the hair and the scalp to relax instantly and to start and restore hair growth.

### SUMMARY OF THE INVENTION

In one aspect of the present invention, a hydrating moisturizer hair therapy oil includes an admixture of black castor oil approximately one-third by weight; coconut oil approximately one-third by weight; Jamaican mango and lime black castor oil approximately one-sixth by weight; tea tree oil approximately one-twelfth by weight; and argon oil approximately one-twenty-fourth by weight, and in certain embodiments biotin oil approximately one-twenty-fourth by weight and chebe powder.

In another aspect of the present invention, a method of hair therapy, includes applying the above-mentioned hydrating moisturizer hair therapy oil that is heated.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention may include a natural hydrating moisturizer hair therapy oil having as components coconut oil, tea tree, organic black castor oil, biotin, argon oil, and Jamaican mango and lime black castor oil extra dark.

Individually, the above-mentioned components by themselves or randomly admixed together do not work because the vitamins and minerals are separated Combining all components at once can help hair and scalp needs when the components listed above are properly mixed together as follows: to make 12 ounces of the hair therapy oil the component parts may be approximately 4 oz. of black castor oil, 4 oz. coconut oil, 2 oz. of Jamaican mango and lime black castor oil, 1 oz. tea tree oil. ½ oz. argon oil, and ½ oz. biotin.

The term “approximately” means within a range plus or minus five percent (5%). By way of example, 1 ounce could range between 0.95 ounces or 1.05 ounces.

All component parts may be necessary to produce the synergetic effect disclosed above. Chebe powder may also be added. A person would use the natural hydrating moisturizer hair therapy oil on post-washed wet and/or dry hair as needed. Whereby the user can grease, oil, moisturize and hydrate their hair and scalp for healthy therapy. The present invention can also be used as a hot oil treatment.

Moreover, the inventor has discovered that the system components, such as but not limited to the natural oils, of the present invention work very well together to provide satisfying results. Specifically, the natural oil gives a fighting chance to regain self-confidence. Certain embodiments of the present invention have many different vitamins and mineral [symbol] properties applicable to local areas on the body for hair growth or dryness. For example, a resulting product of the process of the present invention treats the following: hair loss, alopecia, folliculitis of the scalp, eczema, sensitive skin, dryness of the hair and scalp, cradle cap for babies, stress spot hair loss, and bolding.

Other ways to use the natural hydrating moisturizer hair therapy oil embodied in the present invention is on post-washed wet and/or dry hair as needed in order to grease the hair, moisturize and hydrate their hair and scalp as well as a form of hot oil treatment.

The present invention can also be an additive for shampoos and conditioner to keep hair from stripped from dryness allowing hair to keep moisture. Each ingredient that is used, helps protect hair and scalp from reoccurring damaging damages. All ingredients are necessary to produce all of the plurality of working product or embodiments of the present invention. Chebe powder may also be an added ingredient to help severe damage and faster results.

The systemic components of the present invention may include:
1.coconut oil;
2.tea tree;
3.organic black castor oil;
4.biotin 10000 mcg (50);
5.argon oil;
6.Jamaican mango and lime black castor oil extra dark;
7.Sometimes for chebe powder for extra strength.

The systemic tools of the present invention may include:
1. Melting pot;
2. Measuring cup;
3. Table spoon;
4. Whisk;
5. Teaspoon;
6. Mason.

To make 12 ounces of the hair therapy oil the following arrangement of components may include:
approximately 4 oz (½ cup) of black castor oil;
4 oz (½ cup) coconut oil;
2 oz (4 tsbs) of Jamaican mango and lime black castor oil;
1 oz (2 tsbs). tea tree oil;
1/2 oz (3 tsp); argon oil;
1/2 oz (3 tsbs) is chebe powder.

When adding ingredients, temperature may be at least 140 degrees. Add each ingredients 15 minutes apart to allowing ingredients to infuse. Then set temperature to 100 degrees for 4-6 hours and mix every 30 minutes. Hair serum can be applied to the scalp at room temperature or for a hot oil treatment.

The resulting admixture may be mixed with a melting pot over 4-6 hours depending on the batch size. Stir or mix every 30 minutes to help infuse ingredients.

### Coconut Oil Benefits

To help keep hair follicles healthy and protect the root and strand from breakage, coconut oil Is a natural antioxidants and nutrient that's medium chain fatty acid. Vitamins E, K, and iron are found in coconut oil to help boost growth and re-growth of hair.

Coconut oil helps hair retain its natural protein while reducing the inflammation of the scalp. Now keeping scalp moisturized for the antimicrobial properties that help treat the harmful bacteria of the scalp healing damage open scalp.

Coconut oil keep your scalp hydrated, strengthen hair and nourish the scalp, reducing hair breakage, prevent hair loss, and repair brittle hair, reduces oil buildup. Helps protect against excess heat.

### Tropic Isle Living Jamaican Black Castro Oil Benefits

Jamaican black Castro oil is a healing oil that treat dry and itchy scalp or skin like: eczema, acne, rash, cuts and burn.

Jamaican black Castro oil has antibacterial and anti-fungal properties that are beneficial against folliculitis, dandruff and scalp infections. Richinolic acid content helps increases circulation to the scalp, increase blood flow to the scalp, moisturize, thicken, strengthens and rapidly increasing to improve hair growth, as well as supplying valuable nutrient to the hair follicles. The rich nutrients vitamin E, omega-6 fatty acids, omega 9 fatty acid and ph balance for scalp.

### Tea Tree Oil

Antimicrobial properties to improve scalp health. Helps to unclog hair follicles and nourish your root. Sooth dry and itchy scalp damage hair. Control access oil produce in the scalp that can block hair follicles from preventing hair growth. Thick hair growth and moisturize scalp. Natural anti-fungal and antibacterial properties that can help in maintaining a clean healthy scalp for strong healthy hair follicles. Is an anti-fungal much like ketoconazol that is found in the shampoo Nizoral. Tea tree oil can help as a repellent for lice, and can be used as a hot oil treatment. Mechanism keeping the scalp clean and free of scalp DHT.

### Jamaican Mango and Lime Jamaican Black Castro Oil Extra Dark

Can be used as a natural leave in conditioner that helps deeply conditions the hair and scalp. Seals in moisture to improve hair strength. Helps seal split ends by using as a hot oil treatment as well as moisturizer for dry scalp to regrow hair.

### Argan Oil of Morocco

Alcohol free oil to help stop hair breakage and repair frizz control. Conditioning and absorbs instantly does not leave greasy residue. Leaving hair feeing soft, shining, smelling good and color protection.

Vitamin E minerals and antioxidants that helps with the hair elasticity, restoring and rejuvenate hair back to normal.

### Ultra-Mega Biotin 10,000 mcg

Ultra-mega biotin supplement is a soft gel vitamin B gluten free and have 10,000 mcg. Ultra-mega biotin supports healthy hair growth, skin and nails.

### Chebe Powder

Strength hair, wherein the hair is able to grow without breaking as well as improves hair density, nourish follicles, moisturizes hair, strengthening hair follicles to allow growth healing properties.

Overall the present invention will help with the following: hydrating scalp, moisturize hair and scalp, hair will grow longer, thicker and faster helps, remove sebum build up from hair follicles, regrow hair, improving blood circulation to the scalp, antibacterial treatment for the scalp and anti-inflammatory, relaxation of the scalp, unclog hair follicles, nourished hair scalp and roots, help kills certain bacteria viruses and fungi, may act as a leave-in-conditioner, increase activity of white blood cells to fight germs and folliculitis, provide a source of vitamin E, strengthen hair and follicles, act as a powerful antioxidant, tame frizz, increase shine and softness with less breakage, increase hair elasticity and restore lifeless hair, and improves hair density.

The inventor has discovered that when using the above ingredients separately, does not have the same results or effects as it would if i were to combine ingredients. Each oil is used to serve a purpose to give the proper vitamins and minerals is needed for the scalp and hair to grow and or improve. When combining these ingredients according to the above disclosure, the present invention gives instant relief and therapy to the scalp and or places needed for dryness.

The present invention may be used to grow hair where hair follicles are. Used to apply locally to the infected area or non-infected area for treatment and therapy that promotes strong healthy growth. Hair serum can be applied to the scalp at room temperature shake then apply and or used for hot oil treatment. Hair growth oil enhances follicle and shaft strength to allow natural hair growth on all hair types. Oil helps a person regain confidence and self esteem. Bring back beautiful bouncy natural curls and helps reverse chemical damage follicles. Most of all invention give the hair the therapy to the scalp and hair.

It should be understood, of course, that the foregoing relates to exemplary embodiments of the invention and that modifications may be made without departing from the spirit and scope of the invention as set forth in the following claims.

What is claimed is:

1. A hydrating moisturizer hair therapy oil, comprising an admixture of:
black castor oil approximately one-third by weight;
coconut oil approximately one-third by weight;
Jamaican mango and lime black castor oil approximately one-sixth by weight;
tea tree oil approximately one-twelfth by weight; and
argon oil approximately one-twenty-fourth by weight.

2. The hydrating moisturizer hair therapy oil of claim 1, further comprising biotin oil approximately one-twenty-fourth by weight.

3. The hydrating moisturizer hair therapy oil of claim 1 further comprising chebe powder.

4. The hydrating moisturizer hair therapy oil of claim 2 further comprising chebe powder.

5. A method of hair therapy, comprising:
applying the hydrating moisturizer hair therapy oil of claim 1 that is heated.
